(19) United States
(12) Patent Application Publication
(10) Pub. No.: US 2012/0237623 A1
(43) Pub. Date: Sep. 20, 2012

(54) Topical Compositions Containing Derris Scandens Benth and a Method of Treating Skin

(75) Inventors: Dmitri S. Ptchelintsev, Jersey City, NJ (US); Xiaochun Luo, New City, NY (US)

(73) Assignee: AVON PRODUCTS, INC., New York, NY (US)

(21) Appl. No.: 13/395,682

(22) PCT Filed: Oct. 14, 2010
(86) PCT No.: PCT/US2010/052640

§ 371 Date: Mar. 13, 2012

Related U.S. Application Data
(60) Provisional application No. 61/266,706, filed on Dec. 4, 2009.

(51) Int. Cl. A61K 8/97; A61Q 19/08

(52) U.S. Cl. 424/757

(57) ABSTRACT

Topical compositions and methods of use to treat symptoms of reduced skin elasticity are provided comprising extracts of a. Derris scandens Benth. plant. These compositions are effective in stimulating LOXL-1, activity, inhibiting calcineurin activity, and stimulating glycosaminoglycan synthesis in skin cells and can thus reduce age-related skin damage and improve appearance of fine lines, wrinkles, skin sagging and other symptoms of skin aging and/or skin damage.

### CROSS REFERENCE TO RELATED APPLICATIONS

This application claims priority to International Application Serial No. PCT/US10/52640 filed Oct. 14, 2010, which claims priority U.S. Provisional Patent Application Ser. No. 61/266,706 filed Dec. 4, 2009, the contents of which are hereby incorporated by reference in their entirety.

### FIELD OF INVENTION

The present invention relates to topical compositions containing Derris scandens Benth. (“Derris”) extracts as well as methods of use of such compositions for treating, reversing, and/or preventing signs of skin damage or skin aging, including fine lines and wrinkles in the skin,

### BACKGROUND OF THE INVENTION

The gradual development of facial wrinkles, whether fine surface lines or deeper creases and folds, is an early sign of accumulated skin damage and skin aging, which may be intrinsic and/or caused or accelerated by external factors. For example, premature aging and wrinkling of the skin may be accelerated by excessive exposure to the sun and other damaging elements, overactive facial expression muscles, frequent use of tobacco products, poor nutrition, or skin disorders. Fine surface wrinkles that progress to deeper creases, deepening facial expression due to repeated skin folding, and deep folds which develop with one's maturity are visible changes which may combine to portray a less desirable appearance. Several invasive techniques are available in which substances are injected or implanted in the area of the skin which either temporarily weaken the muscles or act as skin volume fillers. However, invasive techniques are often risky and require the supervision or assistance of a physician, which can be inconvenient and costly, and non-invasive treatments have historically met with only minimal success. Regardless of the cause of facial creases or folds, safe and effective treatments for reduction or elimination of these problems have been exceedingly difficult to achieve.

Skin elasticity is critical for remediating skin damage or skin aging, such as sagging, reduced skin firmness and youthful appearance, and improving the appearance of fine lines and wrinkles. All these are made possible by elastin, a protein polymer that works like a rubber band, repeatedly stretching and contracting without suffering any damage.

Elastin polymers are formed by the cross-linking of tropoelastin monomers. Although there are as many as five enzymes that can catalyze this process, it is unclear exactly how the crosslinking is regulated. One of these enzymes, lysyl oxidase-like 1 (“LOXL1”), has been found to be a key regulator of the renewal of elastin polymer, an extracellular matrix component providing connective tissues, including the skin, with elastic properties. Liu and colleagues have reported that LOXL1 is essential for remodeling elastin fibers. Liu predicted that the enzyme is recruited to sites of elastin by the extracellular-matrix protein fibulin-5, According to Liu, LOXL1 then primes tropoelastin monomers (TE) for incorporation into the larger polymer. Liu found that LOXL1 is necessary to prevent age-related loss of elasticity in tissues such as arteries and lungs (Liu, et al. (2004) Elastic fiber homeostasis requires lysyl oxidase-like 1 protein. Nat. Genet. 36(2):178-82).

Elastin is not believed to be produced past puberty, after which maintenance of the elastin polymers in tissue is regulated by competing activities of renewing (e.g., “anti-elastase”) and degrading (e.g., elastase) the elastin polymer. As one ages, an imbalance in the competing activities occurs, which results in a loss of elasticity in elastin containing tissues. This loss of elasticity in skin can appear as wrinkles in the surface of the skin. Although the exact mechanisms for regulating the renewal and degradation of elastin “anti-elastase” and elastase activity) are unknown, the enzyme, lysyl oxidase-like 1 (“LOXL1”) has been reported as an “anti-elastase” factor, capable of mediating the renewal of elastin fibers by polymerization of tropoelastin monomers (see, e.g., Kaganet al, (2003) J. Cell. Biochem. 88:660-672; and Li et al. (2004) Nat. Genet, 36:178-182). LOXL1 is thus a key regulator of the renewal of elastin polymer in tissue, which provides connective tissues, including the skin, with elastic properties. Accordingly, agents that act to increase LOXL1 transcription and/or translation or LOXL1 activity is believed to increase “anti-elastase” activity, fostering renewal of elastin fibers and effecting an improvement in elasticity of elastin containing tissues, such as the skin.

Noblesse, et al. reported that LOXL1 is present in the dermis and the epidermis of both normal skin and in a skin equivalent model (SE). The ultrastructural localization of lysyl oxidase-like (LOXL) was indicative of its association with elastin-positive materials. The investigators hypothesized that LOXL could have a role in elastic fiber formation (Noblesse E, et al. (2004) Lysyl oxidase-like and lysyl oxidase are present in the dermis and epidermis of a skin equivalent and in human skin and are associated to elastic fibers. J Invest Dermatol. 122(3):621-30).

Pascual and colleagues reported that levels of markers of elastin synthesis, including LOXL1, diminish to a significant extent with age (Pascual, et al. (2008) Down-regulation of lysyl oxydase-like in aging and venous insufficiency. Histol Histopathol. 23(2):179-86).

U.S. Patent Pub. No. 2005/0188427 by Li and Liu describes a method of treating a subject having a condition associated with a loss of elastic fibers, such as loose or wrinkly skin, comprising administering to the subject a therapeutically effective amount of a LOXL1 enhancer, The LOXL1 enhancers are said to be LOXL1 polypeptides or active fragments thereof, or a nucleic acid encoding a LOXL1 polypeptide or active fragment thereof. The LOXL1 enhancers are also said to include small molecules or other therapeutic compounds identified by the screening method described therein.

Calcineurin is a protein phosphatase involved in the activation of Nuclear factor of activated T cells (“NEAT”), a transcription factor. Activation of NEAT transcription factor stimulates T cells involved in calcium trafficking and inflammatory responses. Expression of calcineurin in skin increases intrinsically over time with age.

Topical calcineurin inhibitors, such as a tacrolimus ointment, which is commercially available as PROTORIC®, have been used to treat atopic dermatitis, which is an eczematous skin disease that has typically been treated with topical steroids. A tacrolimus ointment, which is commercially available as PROTOPIC®, has been reported to inhibit calcineurin, which results in suppression of antigen-specific T-cell activation and inhibition of inflammatory cytokine release. (see, e.g., Bekersky et al, 2001, J. Am. Acad. Dermatol, 441:S17-S27). Furue et al. reported that tacrolimus ointment was used as a first-line treatment for the inflammation of atopic dermatitis. (see e.g. Fume, et al. (2006) Dermatol. Ther. 19:118-26). Another commercially available calcineurin inhibitor is pimecrotimus, which is commercially available in a cream and sold as in a cream form, commercially ELIDEL®.

It has been observed that inflammation may have a deleterious effect on the appearance of skin. Hypopigmentation and hyperpigmentation in the skin have been observed post-inflammation. (see Ruiz-Maldonado et al. (1997) Semin Cutan Med Surg. 16(1):36-43; Tomita et al, (1989) Dermatologica. 179 Suppl 1:49-53). Holland et al. observed that certain patients suffering from inflamed acne lesions also demonstrated acne scarring of the skin. (see Holland et al, Semin Cutan Med Surg. 2005 June; 24(2):79-83). Pillai et al. reported that inflammation and the resulting accumulation of reactive oxygen species (ROS) play an important role in the intrinsic and photoaging of human skin in vivo. Pillai et al. reported that the inflammation further activates the transcription of various matrixes degrading metalloproteases, leading to abnormal matrix degradation and accumulation of non-functional matrix components. Pillai et al. observed that the inflammation and ROS cause oxidative damage to cellular proteins, lipids and carbohydrates, which accumulates in the dermal and epidermal compartments, contributing to the aetiology of photoaging. Pillai et al. listed a number of strategies for preventing photodamage caused by this cascade of reactions initiated by UV, which include prevention of UV penetration into skin by physical and chemical sunscreens, prevention/reduction of inflammation using anti-inflammatory compounds (e,g., cyclooxygenase inhibitors, inhibitors of cytokine generation); scavenging and quenching of ROS by antioxidants; inhibition of neutrophil elastase activity to prevent extracellular matrix damage and activation of matrix metalloproteases (MMPs), and inhibition of MMP expression (e.g., by retinoids) and activity (e,g., by natural and synthetic inhibitors). (see et al. (2005) Int J Cosmet Sci. February; 27(1):17-34).

Thornfeldt et al. recommended that skin care regimens using active ingredients that are recommended by physicians who treat mucocutaneous conditions including aging should become more focused on reversing and preventing chronic inflammation. Thornfeldt reported that chronic inflammation appears strongly linked to many preventable and treatable skin diseases and conditions such as visible skin aging. Thornfeldt stated that mucocutaneous inflammation as the final common pathway of many systemic and mucocutaneous diseases including extrinsic aging has been established at the molecular and cellular levels. (see Thornfeldt, C R (2008) J. Cosmet. Dermatol. 7:78-82).

Bissett et at reported that albino hairless mice exposed chronically to suberythemal doses of ultraviolet (UV) radiation displayed an increase in dermal cellularity, including inflammatory cells. In one experiment, Bissett observed that topical hydrocortisone, ibuprofen, and naproxen protected against UVB radiation-induced visible wrinkling, tumor formation, and histological alternations in albino hairless mice. In another experiment, Bissett observed that hydrocortisone and naproxen were effective against UVA radiation-induced visible skin sagging and histological alterations in albino hairless mice. The investigators hypothesized that this data suggested role for inflammation in chronic photodamage. (see Bissett, et al. (1990) Photodermatol. Photoimmunol. Photomed. 7:153-8).

Glycosaminoglycans (GAGs) are long unbranched polymers formed from repeating disaccharide units comprising a variety of hexosamine, hexose or hexuronic acid moieties and sulfates thereof Typically, each disaccharide unit of GAGs consists of a hexosamine moiety (e.g., D-glucosamine or D-galactosamine), or a sulfate thereof, and a hexose (e.g., galactose) or a hexuronic acid moiety (e.g., D-glucuronic or L-iduronic acid), or a sulfate thereof GAGs typically observed in the skin include hyaluronic acid, certain chondroitin sulfates, and dermatan sulfate. In addition, heparin and heparan sulfate have been observed in small amounts in the skin.

GAGs have been considered an important component of extracellular matrix. GAGs can be covalently bound to proteins to form proteoglycans. Proteoglycans are present in all mammalian tissues, including the skin, and is believed to contribute to the growth, preservation and repair of the tissues. It has been reported that GAGs and proteoglycans are involved in the age-related changes in skin mechanical properties, including changes in tissue hydration and resiliency. (see Carrino et al., Arch Biochem Biophys. 2000 Jan. 1; 373(1):91-101; Vogel et at, Z Gerontol, 1994 May-June; 27(3):182-5; Lanir et al., J Biomech Eng. 1990 February; 112(1):63-9). The glycosaminoglycan, hyaluronic acid, has also been reported to be responsible for hydration, nutrient exchange and protection against free radical damage of the skin. (see Wiest et al. J Dtsch Dermatol Ges. 2008 March; 6(3):176-80; Bert et al. Biorheology, 1998 May-June; 35(3):211-9). Wiest et al. reported that in a clinical study an increase in elasticity and turgor was demonstrated following repeated injections with hyaluronic acid. It has also been reported that GAG synthesis declines and the overall GAG content in skin decreases over time as the skin ages. (for example: Smith et al. in J. Invest, Dermatol, 1962, 39, pages 347-350; Fleischmajer et al. in Biochim. Biophys. Acta, 1972, 279, pages 265-275; or Longas et al. in Carhohydr. Res., 1987, 159, pages 127-136).

Derris scandens Benth. (“Derris”) is a woody vine typically found growing throughout Southeast Asia, including Thailand. Specifically, Derris is a climbing branched shrub with a twining habit. It maintains its leaves year-round and is classified as an evergreen, perennial plant. Derris belongs to the Fabaceae plant family and is commonly referred to in English as the “Jewel Vine” and commonly known as “Thaowanprieng” or “Tao-wan-priang” in Thai. A number of Thai folkloric uses have been reported, including uses for pain relief, diuretic, muscle stiffness/pain. (see Chuakul et al., Medicinal Plants in Thailand, Vol II, Bangkok Thailand: Mahidol University, 1997). Chuakul et al, also reported that Derris can impart a number of beneficial pharmacological properties, such as antibacterial, antihistaminic, and hypotensive activities. In addition, it has been reported that an Derris extract may inhibit in vitro activities of eicosanoid, myeloperoxide, and leukotriene B(4), which are activities related to inflammation (see e.g., Laupattarakasem, et al. (2004) Planta Med. 70:496-501 and Laupattarakasem, et al. (2003) J Ethnopharmacol, 85:207-15). In a rat hind paw edema test, a Derris extract was reported as active when administered intraperitoneally but did not produce a significant effect when administered orally. (see Laupattarakasem, et al. (2003) J Ethnopharmacol. 85:207-15). The activity of Derris as a topical treatment to human skin for anti-aging purposes is heretofore unknown.

There remains a need for cosmetic compositions which reduce signs of aging including sagging, reduced elasticity, fine lines and wrinkles, particularly on the skin of the face, neck, hands, etc.

It is therefore an object of the present invention to provide improved compositions and methods of use to improve the appearance of skin, including by reducing the appearance of fine lines and wrinkles.

### SUMMARY OF THE INVENTION

It has been surprisingly found that extracts of Derris are efficacious in reducing, reversing, ameliorating, and/or preventing signs of skin aging caused by decreased skin elasticity, including appearance, depth, or severity of fine lines and/or wrinkles, skin sagging and related signs of aging in skin.

Generally, the compositions and methods are useful for treating any skin condition associate with loss of elastic fibers, typically found in humans. These benefits are believed to arise, at least in part, from the ability of the extracts to stimulate production of LOXL-1, inhibit calcineurin activity, and/or stimulate synthesis of GAGs. Derris extracts can thus potentially reduce the signs of aging, such as wrinkling, discoloration of skin, and reduced elasticity through at least one of these three independent mechanisims.

Based on this discovery, one embodiment provides a method of providing anti-aging benefit to skin comprising applying to an area of the skin, characterized by one or more signs of skin aging, such as wrinkles, fine lines, sagging skin, atrophy, loss of skin resilience or turgor, or loss of skin coloration or tone, a topical composition in a cosmetically acceptable vehicle comprising an extract of Derris in an amount effective to provide an anti-aging benefit to the skin. Such signs of skin aging include without limitation, the following:

(a) treatment, reduction, and/or prevention of fine lines or wrinkles,

(b) reduction of skin pore size,

(c) improvement in skin thickness, plumpness, and/or tautness;

(d) improvement in skin suppleness and/or softness;

(e) improvement in skin tone, radiance, and/or clarity;

(f) improvement in procollagen and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization and/or hydration;

(o) improvement of (e.g., increase in and/or prevention of loss of) skin elasticity and/or resiliency;

(p) reduction in appearance of pigment spots;

(q) treatment, reduction, and/or prevention of skin sagging; and/or

(r) treatment, reduction, and/or prevention of mottled skin appearance.

In one aspect of the invention, a method is provided for preventing, treating or ameliorating wrinkles or fine lines in skin comprising topically applying to the skin in need thereof a composition comprising an extract of Derris and a cosmetically acceptable vehicle.

In another aspect of the invention provides a topical composition for providing an anti-aging benefit to skin comprising an amount of an extract of Derris effective to provide an anti-aging benefit to the skin, and a cosmetically acceptable vehicle. The topical composition may be in the form of a lotion, cream, gel or foam.

These and other aspects of the invention will be better understood by reference to the following detailed description of the invention.

### BRIEF DESCRIPTION OF THE FIGSURES

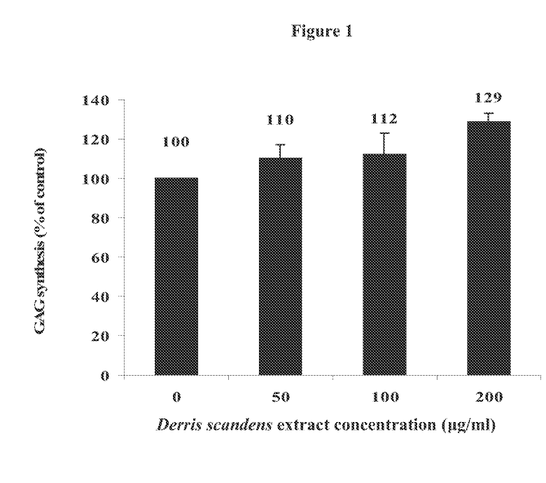

FIG. 1 shows GAGs synthesis as a function of the concentration of Derris extract administered to human fibroblast cells.

### DETAILED DESCRIPTION

In the following description of the invention, it is to be understood that the terms used herein have their ordinary and accustomed meanings in the art, unless otherwise specified. All weights percentages referred to herein are given in terms of “% by weight” or “% wt” of the total composition, which refers to the weight percent of the total formulation after addition of any carriers, solvents, emollients, or other components before application to the skin, unless otherwise indicated,

It has surprisingly been found that an extract of Derris is able to stimulate activity and/or expression of LOXL1, inhibit calcineurin activity, and stimulate synthesis of GAGs. LOXL1 is an essential enzyme necessary for the maintenance and remodeling of elastin in skin. An increase in LOXL1 activity is believed to foster renewal of elastin fibers and effecting an improvement in elasticity of skin Expression of calcineurin in skin increases intrinsically over time with age. Calcineurin is believed to be related to inflammation, which may have a deleterious effect on the appearance of skin Inhibition of calcineurin is expected to provide a decrease in the inflammation contribution to skin aging. GAGs are components of the extracellular matrix and provide for mechanical properties of characteristic of younger skin (e.g., improved elasticity) and for improved hydration of skin. The decreasing amount of GAGs in older skin may be a reason that older skin appears aesthetically inferior to that of younger skin. It is believed that stimulation of GAGs synthesis can provide for alleviation and retardation of the onset of undesirable skin aging characteristics. The topical compositions comprising an extract of Derris according to the invention reduce signs of aging, such as wrinkling, discoloration of skin, and reduced elasticity, through at least one of these three independent mechanisms.

The term “wrinkle” or “wrinkling” refers to both fine wrinkling and coarse wrinkling Fine wrinkling or fine lines refers to superficial lines and wrinkles on the skin surface. Coarse wrinkling refers to deep furrows, particularly deep lines/wrinkles on the face and around the eyes, including of expression lines such as frown lines and wrinkles, forehead lines and wrinkles, crow's feet lines and wrinkles, nasolabial fold and marionette lines and wrinkles. Forehead lines and wrinkles refer to superficial lines and/or deep furrows on skin of the forehead. Crow's feet lines and wrinkles refer to superficial lines and/or deep furrows on skin around the eye area. Marionette lines and wrinkles refer to superficial lines and/or deep furrows on skin around the mouth. Wrinkles can be assessed for number, length, and depth of the lines.

Discoloration of skin includes discrete pigmentation, which is commonly known as pigment spots or “age spots,” and mottled pigmentation. Discrete pigmentation are distinct uniform areas of darker pigment and may appear as brown spots or freckles on the skin. Mottled pigmentation are dark blotches that are larger and more irregular in size and shape than discrete pigmentation. Areas of mottled pigmentation tend to become darker with sun exposure.

Elasticity of the skin refers to the springiness and resilience of skin's ability to regain its original shape and size after deformation. Elasticity of the skin may be evaluated by a pinch test that can either cause deformation by stretching or squeezing the skin

In view of these findings and others, a topical composition comprising an extract of Derris is contemplated to be useful in combating signs of skin damage and skin aging, including reducing fine lines and wrinkles, pigment spots, skin sagging, loss of elasticity, mottled skin appearance, and related signs of aging in skin It is contemplated that a topical composition comprising an extract of Derris may be applied to any surface of the skin, including without limitation, the skin of the face, neck, and/or hands.

The present invention provides compositions for topical application which comprises an effective amount of an extract of Derris to treat, reverse, ameliorate and/or prevent signs of skin damage or skin aging. Such benefits include without limitation, the following:

(a) treatment, reduction, and/or prevention of fine lines or wrinkles,

(b) reduction of skin pore size,

(c) improvement in skin thickness, plumpness, and/or tautness;

(d) improvement in skin suppleness and/or softness;

(e) improvement in skin tone, radiance, and/or clarity;

(f) improvement in procollagen and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization and/or hydration;

(o) increase in skin elasticity and/or resiliency;

(p) reduction in appearance of pigment spots;

(q) treatment, reduction, and/or prevention of skin sagging; and/or

(r) treatment, reduction, and/or prevention of mottled skin appearance.

The compositions of the invention may be applied to skin in need of treatment. That is, skin which suffers from a deficiency or loss in any of the foregoing attributes or which would otherwise benefit from improvement in any of the foregoing skin attributes. Alternatively, the compositions of the invention maybe applied to any surface of the skin, preferably the skin of the face, neck, chest and/or hands, to prevent a deficiency or loss in any of the foregoing attributes.

In certain preferred embodiments the compositions and methods of the invention are directed to the prevention, treatment, and/or amelioration of fine lines and/or wrinkles in the skin. In this case, the compositions are applied to skin in need of treatment, by which is meant skin having wrinkles and/or fine lines. Preferably, the compositions are applied directly to the fine lines and/or wrinkles. The compositions and methods are suitable for treating fine lines and/or wrinkles on any surface of the skin, including without limitation, the skin of the Ike, neck, and/or hands.

One component of the invention is a botanical component derived from the Derris plant, preferably derived directly from the Derris plant. The botanical component may be in a pure form, a semi-pure form, or unpurified form. The Derris botanical component may be in the form of an extract obtained by solvent extraction. Preferably, the Derris botanical component is obtained by an organic solvent extraction. Specifically, a polar organic solvent may be used. More preferably, the Derris botanical component is obtained by an aqueous-organic solvent extraction.

Suitable organic solvents include, but are not limited to, alcohols (such as methanol, ethanol, propanol, butanol and the like); glycols; ethers (such as diethyl ether, dipropyl ether, and the like); esters (such as butyl acetate, ethyl acetate, and the like); ketones (such as acetone, ethyl methyl ketone, and the like); organic acids including acetic acid, and the like; dimethyl sulfoxide; acetonitrile; isopropanol; dichloromethane; chloroform; hexane; xylene; petroleum ether; other organic solvents; and combinations thereof. Other suitable solvents include water, physiological saline, phosphoric acid buffer and phosphate butler saline and the like.

Solvent extraction involves collecting the raw materials from the Derris plant that contain the desired constituent(s), such as seeds, needles, leaves, roots, bark, cones, stems, rhizomes, callus cells, protoplasts, organs and organ systems, and meristems. Preferably, the extract may be obtained from the steins of the Derris plants. In certain embodiments, raw material collected from the Derris plant may be dried to reduce the raw material's water content. The raw material may be dried by a number of different means, such as, for example, air-dried, oven-dried, rotary evaporated under vacuum or lyophilized. In a preferred embodiment, the raw material comprises dried stems of the Derris plant.

Briefly, the organic solvent extraction method involves washing and extracting the raw material using an organic solvent. An extracting machine may be used for solvent extraction as is well known in the field. In certain embodiments, the Derris plant material is ground to small particle sizes, and then put into an extracting machine through an inlet for the raw material by a measurable charging machine. The plant raw material is pushed in the extracting machine by a thruster, which slowly moves the plant raw material forward. A solvent may be added into the machine through a solvent inlet at the top of a waste discharge outlet. Due to the difference in gravity and equilibrium, the solvent flows toward the raw material inlet, soaks the raw material and flows out from the opposite side of the solvent inlet. Since the raw material and the solvent continuously move in opposite directions against each other, the raw material is constantly immersed in a solution that contains a low-concentration of extract. As a result of equilibrium, high yield of plant constituent(s) may be achieved by continuously extracting the plant material against the low-concentration solution.

An extraction time suitable to extract the plant constituents is used, typically between about 1-12 hours is suitable, and more preferably is between about 2-8 hours, and most preferably is between about 3-6 hours. The temperature of extraction is between about 25° C.-90° C., preferably between about 35° C.-70° C., and more preferably between about 50° C.-60° C. The collected extract is then fine-filtered to remove debris, and may be used directly, or is concentrated, for example by distilling the solvent or by other conventional processing. The solution of extract actives may be rotary evaporated under vaccuum or lyophilized. A typical extract actives content is above about 25%, preferably above 50%, and the extract can also be provided in powder form, including a lyophilized powder.

Similarly, aqueous-organic solvent extraction involves initially collecting raw materials from a plant containing the desired constituent(s), such as seeds, needles, leaves, roots, bark, cones, stems, rhizomes, callus cells, protoplasts, organs and organ systems, and meristems of the plant, which may be ground into small particle sizes. The ground plant material is soaked in aqueous solution that is acidic or alkaline, depending on the solubility and stability of the desired extract under acidic or alkaline (basic) conditions. For extraction under acidic conditions, an acid such as hydrochloric acid or sulfuric acid is added to water, e.g., at a concentration of about 3% (w/v). For extraction under alkaline conditions, an alkali such as sodium hydroxide or sodium carbonate is added to water. The extraction time and temperature of extraction are typically similar to that used in the organic solvent extraction method described above.

Preferably, the Derris extract is obtained by extracting Derris sterns with water, ethanol, or a mixture thereof. The preferred solvent systems will comprise from about 10% by volume to about 90% by volume of ethanol and from about 10% by volume to about 90% by volume of water. More typically, the solvent system will comprise from about 25% by volume to about 75% by volume of ethanol and from about 25% by volume to about 75% by volume of water. Particularly good results are obtained with a solvent system comprising from about 45% by volume to about 55% by volume of ethanol and from about 45% by volume to about 55% by volume of water, with a 50:50 mixture (by volume) of ethanol and water being preferred.

The extract is then collected and fine-filtered to remove debris. Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, concentrated or dried. Alternatively, organic solvent may then be added to the neutralized solution to transfer the extract from an aqueous phase to an organic phase. Examples of such organic solvents include, but are not limited to, ethanol, isopropanol, butanol, pentanol, hexanol and xylene. The extract comprising the transferred extract actives dissolved in organic solvent may be used directly, used as a concentrate, or dried. The solution of extract actives may be rotary evaporated under vaccuum or lyophilized. The extract can be provided as a liquid or a solid, preferably in the form of a dried powder, including a lyophilized powder.

Suitable extraction processes are disclosed in PCT Publications WO03/079816 (describes a process for the preparation of tomato extracts with high content in lycopene), WO04/014404 (describes a process (or the preparation of an Echinacea angustifolia extract) and WO04/014958 (describes extracting a polysaccharide of Echinacea angustifolia roots), all of which are herein incorporated by reference in their entirety.

Different plants containing different constituents may be mixed and extracted together with the Derris plant matter. This process of mixed extraction may preferably be used for extracting those plants containing constituents having similar solubility as those in Derris the solvent used for extraction, such as ethanol. The mixture of extracts may be concentrated and stored in an appropriate solvent.

The compositions according to the invention can be formulated in a variety of forms for topical application and will comprise from about 0.0001% by weight to about 90% by weight of the extract of Derris. Across this range, it is believed that the compositions will stimulate activity and/or expression of LOXL1, inhibit calcineurin activity, and/or stimulate synthesis of GAGs. The composition may comprise from about 0.001% by weight to about 25% by weight of the extract of Derris Preferably, the composition comprises from about 0.01% by weight to about 10% by weight of the extract of Derris. Within the preferred range, the composition may comprise a Den-is extract within a range from about 0.1%, 0.25%, 0.5%, 0.75% or 1% by weight up to 5%, 7.5% or 10% by weight of the total composition.

The above amounts are refer to an “active amount” of the Derris extracts. The term “active amount” refers to the amount of Dernis extract absent diluent, solvent, or any other ingredient added for bulk. An “amount effective” or an “effective amount” to provide a particular anti-aging benefit to the skin refers to the “active amount” of extract required to provide a clinically measurable improvement in the particular manifestation of skin aging when applied for a time sufficient to provide a clinically measurable improvement in the particular manifestation of skin aging.

In another embodiment, the Derris extract as used herein, also includes “synthetic” extracts, i.e., various combinations of known Derris plant components and/or constituents that are combined to substantially mimic the composition and/or activity of a Derris plant extract of natural origin. Most preferably, the synthetic extracts have substantially the same number of active components as a natural Derris plant material. The correspondence of the numerical incidence of actives between the synthetic extracts and the natural Derris plant material may also be described in terms of “percent commonality.” The synthetic extract has about 50 percent or more commonality to the chemical composition of a plant or natural extract. In other words, the synthetic extract has about 50 percent or more of the active ingredients found in the plant or a natural extract. More preferably, the chemical composition of the synthetic extract has about 70 percent or more commonality to the chemical composition of a plant or a natural extract. Optimally, a synthetic extract has about 90 percent or more commonality to the chemical composition of a plant or a natural extract.

The composition may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. Preferably the composition is formulated as a lotion, cream, ointment, gel, and stick. More preferably the composition is formulated as a lotion or a cream. Additionally, targeted delivery and/or penetration enhancement may be achieved by iontophoresis. The foregoing product forms will preferably comprise an emulsion and one or more ingredients in addition to the extract and the vehicle, such as one or more ingredients having a beneficial effect on the skin.

The compositions can include a cosmetically acceptable vehicle. Such vehicles may take the form of any known in the art suitable for application to skin and may include water (e.g., deionized water); vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, preferably organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures of the foregoing.

In one embodiment, the compositions of this invention comprise a cosmetically acceptable vehicle (diluent or carrier) by which is meant that it is compatible with human skin. The compositions may comprise an aqueous phase, an oil phase, an alcohol, a silicone phase or mixtures there of. The cosmetically acceptable vehicle may comprise an emulsion. Non-limiting examples of suitable emulsions may include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant.

The oil phase of the emulsion preferably has one or more organic compounds, including emollients; humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

The compounds suitable for use in the oil phase include without limitation, vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; and the like. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils include those having 6-20 carbon atoms, more preferably 10-16 carbon atoms. Representative hydrocarbons include decane, dodecane, tetradecarte, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99A™ are also contemplated to be suitable. Various commercially available C₁₆ isoparaffins, such as isohexadecane (having the tradename Permethyl®) are also suitable. Examples of preferred volatile hydrocarbons include polydecanes such as isododecane and isodecane, including for example, Permethyl-99A (Presperse Inc.) and the C₇-C₈ through C₁₇-C₁₅ isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, polyethylene waxes, Fischer-Tropsch waxes, beeswax, microcrystaline wax, silicone waxes, fluorinated waxes, and any combination thereof.

Non-limiting emulsifiers included emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. The preferred emulsifiers include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following; sorbitan esters; polyglyceryl-3-diisostearate; C₁₀₋₃₀ alkyl acrylate crosspolymer; Dimethicone PEG-7 isostearate, acrylamide copolymer; mineral oil; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI ingredient Dictionary and Handbook 11th Edition 2006, the disclosure of which is hereby incorporated by reference.

These emulsifiers typically will be present in the composition in an amount from about 0.001% to about 10% by weight, in particular in an amount from about 0.01% to about 5% by weight, and more preferably, from about 0.1% to about 3% by weight.

The oil phase may comprise one or more volatile and/or non-volatile silicone oils. Volatile silicones include cyclic and linear volatile dimethyisitoxane silicones. In one embodiment, the volatile silicones may include cyclodimethicones, including tetramer (D4), pentamer (D5), and hexamer (D6) cyclomethicones, or mixtures thereof. Particular mention may be made of the volatile cyclomethicone-hexamethyl cyclotrisiloxane, octamethyl-cyclotetrasitoxane, and decamethyl-cyclopentasitoxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics”, Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (GE. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few,

Non-volatile silicone oils will typically comprise polyalkylsiloxanes, polyarylsiloxanes, polyalkylarylsiloxanes, or mixtures thereof. Polydimethylsitoxanes are preferred non-volatile silicone oils. The non-volatile silicone oils will typically have a viscosity from about 10 to about 60,000 centistokes at 25° C., preferably between about 10 and about 10,000 centistokes, and more preferred still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted will various functional groups such as alkyl, aryl, amine groups, vinyl, hydroxyl, haloalkyl groups, alkylaryl groups, and acrylate groups, to name a few.

The water-in-silicone emulsion may be emulsified with a nonionic surfactant (emulsifier) such as, for example, polydiorganosilaxane-polyoxyalkylene block copolymers, including those described in U.S. Pat. No. 4,122,029, the disclosure of which is hereby incorporated by reference. These emulsifiers generally comprise a polydiorganosiloxane backbone, typically polydimethylsiloxane, having side chains comprising -(EO)ₘ- and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SIL WET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. Preferred examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in particular in an amount from about 0.01% to about 5% by weight, and more preferably, below 1% by weight.

The aqueous phase of the emulsion may include one or more additional solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like.

The oil-containing phase will typically comprise from about 10% to about 99%, preferably from about 20% to about 85%, and more preferably from about 30% to about 70% by weight, based on the total weight of the emulsion, and the aqueous phase will typically comprise from about 1% to about 90%, preferably from about 5% to about 70%, and more preferably from about 20% to about 60% by weight of the total emulsion. The aqueous phase will typically comprise from about 25% to about 100%, more typically from about 50% to about 95% by weight water.

The compositions may include liposomes. The liposomes may comprise other additives or substances and/or may be modified to more specifically reach or remain at a site following administration.

in one embodiment, the composition of the invention comprising an extract of Derris may have a pH between about 1 and about 8. In certain embodiments, the pH of the composition will be acidic, i.e., less than 7.0, and preferably will be between about 2 and about 7, more preferably between about 3.5 and about 5.5.

The composition may optionally comprise other cosmetic or skin actives and/or excipients. Preferably, the other cosmetic actives and excipients are not strong oxidizing agents and do not have strong oxidizing potential. More preferably, the composition does not include either an organic or an inorganic peroxide.

Suitable other cosmetic or skin actives and excipients include, for example, fillers, essential oils, emulsifying agents, antioxidants, anti-inflammatory agents, surfactants, film formers, chelating agents, gelling agents, thickeners, emollients, humectants butylene glycol, propylene glycol, Methylgluceth-20, and glycerin), moisturizers, vitamins, minerals, plasticizers, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants (including pigments and pearlescent agents), depigmenting agents, hypopigmenting agents, pH adjusters, preservatives (e.g., DMDM Hydantoin/Iodopropynylbutylcarbonate), stabilizers, pharmaceutical agents, photostabilizing agents, neutralizers (e.g., triethanolamine) and mixtures thereof. In addition to the foregoing, the cosmetic compositions of the invention may contain any other active or compound for the treatment of skin disorders.

Colorants may include, for example, organic and inorganic pigments and pearlescent agents. Suitable inorganic pigments include, but are not limited to, titanium oxide, zirconium oxide and cerium oxide, as well as zinc oxide, iron oxide, chromium oxide and ferric blue. Suitable organic pigments include barium, strontium, calcium, and aluminium lakes and carbon black. Suitable pearlescent agents include mica coated with titanium oxide, with iron oxide, or with natural pigment.

Various fillers and additional components may be added. Fillers are normally present in an amount of about 0 weight % to about 20 weight %, based on the total weight of the composition, preferably about 0.1 weight % to about 10 weight %. Suitable fillers include talc, silica, zinc stearate, mica, kaolin, nylon (in particular orgasop powder, polyethylene powder, Teflon, starch, boron nitride, copolymer microspheres such as Expancel (Nobel Industrie), Polytrap (Dow Corning), and silicone resin microbeads (Tospearl from Toshiba).

In one embodiment of the invention, compositions comprising a Derris extract may include skin actives such as, but are not limited to, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, salicylic acid, salicylates, and advanced glycation end-product (AGE) inhibitors.

In a specific embodiment, the composition may comprise at least one additional botanical, such as, for example, a botanical extract, an essential oil, or the plant itself. Suitable botanicals include, without limitation, extracts from Abies pindrox, Acacia catechu, Anogeissus latifolia, Asmunda japonica, Azadirachta indica, Butea frondosa, Butea monosperma, Cedrus deodara, Emblica officinalis, Ficus benghalensis, Glycyrrhiza glabra, Ilex purpurea Hassk, Innula racemosa, Ligusticum chiangxiong, Ligusticum lucidum, Mallotus philippinensis, Mimusops elengi, Morinda citrifolia, Moringa oleifera, Naringi crenulata, Nerium indicum, Psoralea corylifolia, Stenoloma chusana, Terminalia bellerica, tomato glycolipid and mixtures thereof.

The composition may comprise additional active ingredients having anti-aging benefits, as it is contemplated that synergistic improvements may be obtained with such combinations. Other suitable anti-aging components include, without limitation, certain botanicals, such as Butea Frondosa extract, thiodipropionic acid (TDPA), retinoids, hydroxy acids (including alpha-hydroxyacids and beta-hydroxyacids), salicylic acid, salicylates, and glycolic acid, to name a few.

Suitable hydroxy acids include, for example, glycolic acid, lactic acid, malic acid, tartaric acid, citric acid, 2-hydroxyalkanoic acid, mandelic acid, salicylic acid and alkyl derivatives thereof, including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2-hydroxy-3-methyoxybenzoic acid.

Exemplary retinoids include, without limitation, retinoic acid (e.g., ail-trans or 13-cis) and derivatives thereof, retinol (Vitamin A) and esters thereof, such as retinol palmitate, retinol acetate and retinol propionate, and salts thereof.

In another embodiment, the topical compositions of the present invention may also include one or more of the following: a skin penetration enhancer, an emollient, a skin plumper, an optical diffuser, a sunscreen, an exfoliating agent, and an antioxidant.

An emollient provides the functional benefits of enhancing skin smoothness and reducing the appearance of fine lines and coarse wrinkles. Examples include isopropyl myristate, petrolatum, isopropyl lanolate, silicones (e.g., methicone, dimethicone), oils, mineral oils, fatty acid esters, cetyl ethylhexanoate, C₁₂₋₁₅ alkyl benzoate, isopropyl isostearate, diisopropyl dimer dillinoeate, or any mixtures thereof. The emollient may be preferably present from about 0.1 wt % to about 50 wt % of the total weight of the composition.

A skin plumper serves as a collagen enhancer to the skin. An example of a suitable, and preferred, skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

An optical diffuser is a particle that changes the surface optometrics of skin, resulting in a visual blurring and softening of, for example, lines and wrinkles. Examples of optical diffusers that can be used in the present invention include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. When present, the optical diffuser may be present from about 0.01 wt % to about 20 wt % of the total weight of the composition.

A sunscreen for protecting the skin from damaging ultraviolet rays may also be included. Preferred sunscreens are those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alphahydroxyacids, betahydroxyacids, oxaacids, oxadiacids, and their derivatives such as esters, anhydrides and salts thereof. A preferred exfoliating agent is glycolic acid. When present, the exfoliating agent may comprise from about 0.1 wt % to about 80 wt % of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; alpha-hydroxyacids; beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives (e.g., tocopheryl acetate); uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may comprise an antioxidant preferably from about 0.001 wt % to about 10 wt %, and more preferably from about 0.01 wt % to about 5 wt %, of the total weight of the composition.

Methods of reducing visible signs of skin damage or aging, such as, for example, appearance of fine lines in the skin, wrinkles, splotchiness and skin discolorations, are provided that comprise topically applying an effective amount of an extract of Derris to the skin of an individual in need thereof. The method provides for stimulating LOXL-1 activity, such as increasing expression of LOXL-1, inhibiting calcineurin activity, and/or stimulating synthesis of GAGs,

Methods of treating symptoms of reduced skin elasticity are also provided that comprise topically applying effective amounts of an extract of Derris to the skin of an individual in need thereof. Typically, the extract of Derris is provided in the form of compositions comprising a cosmetically acceptable vehicle, by which is meant a vehicle compatible (e.g., safe) with application to human skin.

Reduced skin elasticity is thought to be due to a loss of elastin fibers in the skin. The method provides a maintenance of skin integrity and reduction in the appearance fine lines and wrinkles, such as reduction in the depth of the fine lines and wrinkles, an improvement in skin tone and coloration, a reduction in skin sagging and atrophy, improved resilience and turgor, which refers to the skirt density or cushion and can be evaluated by inching the skin and observing how much tissue comes up. The host is typically suffering from one or more of these symptoms.

The effect of a composition on the formation or appearance of fine lines and wrinkles can be evaluated qualitatively, e.g., by visual inspection, or quantitatively, e.g., by microscopic or computer assisted measurements of wrinkle morphology. Preferably, wrinkle morphology is quantitatively analyzed, e.g., the number, depth, length, area, volume and/or width of wrinkles per unit area of skin are measured.

The compositions ideally increase LOXL1 activity in the skin, inhibit calcineurin and stimulate GAGs synthesis in skin fibroblast cells. The skin damage that can be improved or treated with the compositions of the invention include any signs of reduced skin elasticity such as fine lines and/or wrinkles, fragile or thinning skin, sagging skin, lack-luster skin, fatigued skin, dry skin, skin sensitivity, dark eye circles, puffy skin, irregular skin pigmentation, and melasma.

Topically applying compositions of the present invention to the skin can enhance and improve the aesthetic appearance of skin. This method is particularly useful for treating signs of skin photo- and intrinsic aging, including fine lines and wrinkles, skin tone and coloration, skin sagging and atrophy, enlarged pores, skin thinning, decreased resilience and turgor.

Topically applying compositions of the present invention to the skin can enhance and improve the aesthetic appearance of skin by, among other improvements, decreasing skin fragility; preventing and reversing deterioration of elastin; preventing skin atrophy; (promoting/accelerating cell turnover; improving skin firmness/plumpness; improving skin texture; decreasing length and/or depth of fine lines and wrinkles; preventing formation of fine lines and wrinkles; improving skin tone; enhancing skin thickness; restoring skin luster; minimizing signs of fatigue; reducing skin dryness; reducing skin itchiness; reducing skin redness; reducing sensitivity to chemical, mechanical or radiation impact; reducing propensity of the skin to flush and blush; reducing dark circles and puffiness in the periorbital eye area; reducing frown lines on the forehead and laugh lines around the mouth; increasing cell proliferation; decreasing the extent and/or duration of bruising visible after physical trauma; reducing blotchiness and irregular skin pigmentation; treating or ameliorating melasma; treating or ameliorating skin hyperpigmentation; treating or ameliorating foliculitis barbae and ingrown hair bumps and after-shave nicks and irritation; treating or ameliorating dermatitis, psoriasis and other skin conditions affiliated with or caused by acute, subacute or subchronic inflammation; and enhancing overall skin health.

The invention also provides a method for treating aging skin by topically applying a composition comprising the inventive composition over the affected area for a period of time sufficient to reduce, treat or ameliorate, dermatological signs of reduced skin elasticity.

In one embodiment, a method of treating wrinkles of the skin (e.g., on the face, neck, hands) comprises topically applying a cosmetic composition comprising an extract of Derris to the wrinkle in an effective amount and for a time sufficient to reduce the severity or depth of the wrinkle.

In another embodiment, a method of preventing damage of the skin (e.g., on the face, neck, hands) comprises topically applying a cosmetic composition comprising an extract of Derris to undamaged skin in an effective amount to retard skin damage or skin aging. The skin damage may include, for example. formation of fine lines and wrinkles, pigment spots, skin sagging, loss of elasticity, and mottled skin appearance.

In another embodiment, a method of treating sagging of the skin (e.g., on the face or neck) comprises topically applying a cosmetic composition comprising an extract of Derris to the sagging skin in an effective amount and fir a time sufficient to reduce the degree of sagging.

In another embodiment, a method of treating thin skin comprises topically applying a cosmetic composition comprising an extract of Derris to an thin skin in an effective amount and for a time sufficient to thicken the skin.

“Thin skin” is intended to include skin that is thinned due to chronological aging, menopause, or photo-damage. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in patients that have not manifested signs of skin aging, most commonly in individuals under 25 years of age. The method may also reverse or treat signs of aging once manifested as is common in patients over 25 years of age.

The composition may be applied to the skin for a period of time sufficient to improve the aesthetic appearance of the skin. The composition may be applied to the skin from 1 time, 2 times, 3 times, 4 times, 5 times, 6 times or more per day for as long as is necessary to achieve desired results. The treatment regiment may comprise daily application for at least one week, at least two weeks, at least four weeks, at least eight weeks, or at least twelve weeks. Alternatively, the treatment regiment may comprise chronic application of the composition to skin. The method includes treatment of skin changes associated with both intrinsic skin aging and skin aging caused by exposure to external insults. The method is contemplated to be useful for the treatment of UV damaged skin.

### EXAMPLES

### Example 1

### Preparation of Derris scandens Benth. (“Derris”) Extract

Stems of Derris were used for the extraction. 250 g of stems of Derris was chopped into approximately 5 cm long pieces and dried in the electric oven at 60° C. for 2-3 days. The dried material was extracted with a solvent (EtOH/H₂O 50/50 v/v) three times. For example, 1 liter of the solvent can be used to extract at 37° C. for 12 hrs at 150 rpm.

The volatile compound (i.e. ethanol) is removed under vacuum concentration in a rotary evaporator at 40-50° C. For example, three liters of total extract is reduced to 150 ml at the end of the distillation and is diluted with pure water to 1500 ml and allowed to stand at 4° C. for 12 hrs. The dilution is then centrifuged to remove residue and insoluble matter. The solution is then subjected to extraction with hexane (2×0.5 volume) in order to remove possible traces of toxins such as pesticides. For example 1500 ml of extract solution is treated in separation funnel with 750 ml of hexane. The process is repeated 2 times. The hexane layer is discarded and the aqueous layer is retained for further extraction steps.

The dry matter content of homogeneous solution is checked by sampling and lyophilization. After that, charcoal is added to the defatted solution in order to decolorize and deodorize it. The charcoal is added at 10% by weight vs the total dry matter content of the solution. For example, 1500 ml of solution with a total dry matter content of 170 g requires addition of 17 g of charcoal, followed by stirring at 50° C. for 1 hr and a double filtration through paper filters.

The solution is then fractionated by extraction with water-saturated n-butanol 3×0.5 volume. For example, for 1500 ml of decolorized clear solution, 2,250 ml of water-saturated butanol is prepared and divided into three parts of 750 ml each. The decolorized clear solution is then treated with these portions three times in a separatory funnel. If the separation of layers is not clear, the separation is allowed to continue for 10-12 hours and can be slightly heated with warm air at the end.

The butanolic extracts are pooled together, water is added (same volume) and the solution is concentrated in rotary evaporator under vacuum. When the distillation stops, water is added again and the mixture is concentrated again. The process is then repeated the third time and lyophilized. The aqueous layer is concentrated by rotary evaporation under vacuum to remove butanol. When distillation stops, the aqueous solution is lyophilized. The obtained dry powder is an “extract.”

As noted in the remaining specification, modifications and adaptations of this extraction process are possible, particularly during a scale-up to larger volumes for production.

### Example 2

### Stimulation of LOXL1 Activity

The effect of administering a Derris extract on the expression of LOXL1 in human fibroblasts was investigated using a luciferase-reporter system.

Vector construction, transfection and expression: The promoter region of the LOXL1 gene is isolated and cloned into the pGL3 Luciferase reporter plasmid (Promega) according to manufacturer's instructions. The LOXL1/pGL3 vector and the control vector pRL-NULL (Promega), are co-transfected into the human fibrosarcoma line HT1080 using LipofectAMINE™ LTX Reagent (Invitrogen) according to manufacturer's directions. Transfected cells are allowed to recover for 24 h. The culture medium is then replaced with fresh medium containing extracts of Derris at various concentrations, and the transfected cells cultured for an additional 24 h. The cultures are subsequently washed with Phosphate Buffered Saline (PBS) and exposed to 100 μl cell lysis buffer/25 cm² culture area and gently shaken at room temperature for 30 min. The culture flasks containing the cell lysate are then immediately placed at −80° C.

Determination of reporter activity: The activity of the reporter gene, firefly luciferase, is determined according to the manufacturer's instructions (Dual-Luciferase® Reporter Assay System, Promega). Briefly, the activity of the reporter gene is determined relative to that of a control vector encoding a second luciferase gene, that from Renilla reniformis. The relative activities of the genes from test and control cultures are compared for a determination of percent modification of regulatory sequence activity.

Results: Because pGL3 vectors lack the necessary promoter regions to regulate the luciferase gene, the expression of this gene is controlled by the cloned regulatory elements, in this case the regulatory elements of the LOXL1 gene. In triplicate tests, the addition of 0.004 wt % of an extract of Derris was found to increase expression of the reporter gene by 50% (p<0.05, compared to vehicle control), indicating an increased expression of the regulatory elements of the LOXL1 gene. Therefore, Derris is indicated as a positive regulator LOXL1 expression, suggesting secondary effects on elastin renewal.

### Example 3

### Inhibition of Calcineurin Activity

An ex-vivo phosphatase assay was used to evaluate the ability of a Derris extract to modulate calcineurin activity.

Ex-vivo phosphatase assay: The modulation of calcineurin activity is monitored using a DiFMUP (6,8-difluoro-7-hydroxy-4-methylcoumarin phosphate)phosphatase assay (see, e.g., Wegner et al., 2007, Methods Mol. Biol. 365:61-69, hereby incorporated by reference in its entirety). Dephosphorylation of DiFMUP leads to the formation of a highly fluorescent and stable product: Di4MU. Varying concentrations of the extract are added to a reaction buffer, typically consisting of 50 mM Tris-HCl, pH 7.4, 0.0125% Bovine Serum Albumin (BSA), 0.1 mM CaCl, 400 U/ml calmodulin and 1 mM NiCl. The reaction buffer mixture is incubated at 37° C. for 30 min. DiFMU substrate is then added to a concentration of 10 μM, and the mixture returned to 37° C. incubation for a further 15 min. Fluorescence intensity is determined on a Spectrofluorometer.

Results: Derris extract was found to inhibit calcineurin activity in a dose dependent manner, demonstrating an inhibitory effect of 103% at a concentration of 1% weight/volume and an inhibitory effect of 49% at a concentration of 0.1% concentration. Derris extract did not exhibit any effect when tested at a concentration of 0.01% weight/volume. The IC₅₀ of the compound was estimated as 0.106% weight/volume. The results suggest that topical application of compositions comprising a Derris extract will result in improvements of symptoms associated with inflammatory skin conditions.

### Example 4

### Measurement of Glycosaminoglycan Synthesis

The effect of administering a Derris extract on the synthesis of GAG in human fibroblasts was investigated.

Generally, techniques to measure glycosaminoglycan synthesis are described Barbosa, et al. (2003) Glycobiology 13:647-653. Briefly, fibroblasts are plated, for example in 24-well plates, and grown in Dulbecco's Modified Eagle Medium (DMEM) supplemented with 10% Fetal Bovine Serum (PBS), 1xAAS and an extract of Derris at various concentrations (i.e., 50 μg/ml, 100 μ/ml, and 200 μg/ml). Samples of cultures are withdrawn, cells are washed with PBS and trypsinized (80 ml/well). DMEM with 10% PBS and 1x AAS (400 ml/well) is used to inactivate trypsin and this solution transferred to 1.5 ml Eppendorf tubes and centrifuged at 2000 r.p.m. in a microcentrifuge for 2 min. Following removal of supernatant, the cells are digested with proteinase K (50 mg/ml in 100 mM K₂HPO₄ pH 8.0; 400 ml/tube) at 56° C. overnight. Proteinase K is inactivated at 90° C. for 10 min. The prepared samples are stored at −20° C. until used for sulfated GAG quantification with Blyscan Assay, and DNA measurement with PicoGreens Quantitation Reagent, according to the manufacturer's instructions.

Results: The results of the GAG quantification of in samples of fibroblast cells were compared against a control sample where the culture medium did not contain an extract of Derris and expressed as a percentage of the control sample as shown in FIG. 1. The results indicated that an extract of Derris stimulates synthesis of GAG in fibroblast cells. As shown in FIG. 1, as the concentration of extract increased, the amount of GAG synthesized by the fibroblast cells steadily increased.

### Example 6

### Exemplary Compositions

Cosmetic compositions comprising an extract of Derris for topical application to the skin are provided in Table 1.

TABLE 1

| | Composition: | | | |
|---|---|---|---|---|
| | 1 | 2 | 3 | 4 |
| Components | Weight % | | | |
| Derris scandens extract | 2 | 0.2 | 0.1 | 0.01 |
| Acrylates/C10-30 Alkyl Acrylate | 1 | 1 | 1 | 1 |
| Crosspolymer | | | | |
| Cetyl Ethylhexanoate | 10 | 10 | 10 | 10 |
| C12-15 Alkyl Benzoate | 3.9 | 3.9 | 3.9 | 3.9 |
| Isopropyl Isostearate | 3 | 3 | 3 | 3 |
| Diisopropyl dimer dillinoleate | 0.1 | 0.1 | 0.1 | 0.1 |
| Tocopheryl acetate | 0.5 | 0.5 | 0.5 | 0.5 |
| Butylene glycol | 2 | 2 | 2 | 2 |
| Propylene glycol | 1 | 1 | 1 | 1 |
| Dimethicone PEG-7 isostearate | 0.5 | 0.5 | 0.5 | 0.5 |
| Methyl gluceth-20 | 0.5 | 0.5 | 0.5 | 0.5 |
| Triethanolamine | 1 | 1 | 1 | 1 |
| Acrylates/acrylamide copolymer/ | 1.5 | 1.5 | 1.5 | 1.5 |
| mineral oil | | | | |
| DMDM Hydantoin/Iodopropynylbutyl- | 0.4 | 0.4 | 0.4 | 0.4 |
| carbonate | | | | |
| Deionized water | q.s. | q.s. | q.s. | q.s. |
| Total: | 100 | 100 | 100 | 100 |

These compositions are believed to be effective to treat, reverse, ameliorate and/or prevent signs of skin aging, specifically, the compositions are believed to reduce the appearance of fine lines and wrinkles in the skin. The compositions of Table 1 are applied to skin in need of treatment, by which is meant skin in need of an anti-aging benefit, and in particular skin having wrinkles and/or fine lines. The cosmetic compositions may be applied directly to the fine lines and/or wrinkles. The exemplary compositions may be applied to treat, reverse, ameliorate and/or prevent fine lines and/or wrinkles on any surface of the skin, including without limitation, the skin of the face, neck, and/or hands.

The cosmetic compositions are applied to the skin, fine line and/or wrinkle one, two or three times daily for as long as is necessary to achieve desired anti-aging results, which treatment regiment may comprise daily application for at least one week, at least two weeks, at least four weeks, at least eight weeks, or at least twelve weeks. Alternatively, the exemplary cosmetic compositions may be used in chronic treatment of the skin, fine line and/or wrinkle.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended along with the full scope of equivalents to which such claims are entitled.

1. A method of providing anti-aging benefit to skin comprising:
applying to an area of the skin, characterized by one or more signs of skin aging, a topical composition in a cosmetically acceptable vehicle comprising an extract of Derris scandens Benth. in an amount effective to provide an anti-aging benefit to the skin

2. The method of claim 1, wherein said extract is present in an amount sufficient to increase expression of LOXL1, inhibit calcineurin activity, or stimulate glycosaminoglycan synthesis.

3. The method of claim 1, wherein said extract is present in an amount from 0,0001 weight % to 90 weight % based on total weight of the composition.

4. The method of claim 3, wherein said extract is present in an amount from 0.001 weight % to 25 weigh % based on the total weight of the composition.

5. The method of claim 4, wherein said extract is present in an amount from 0.01 weight % to 10 weight % based on the total weight of the composition.

6. The method according to claim 1, wherein said one or more signs of skin aging includes wrinkles and/or fine lines.

7. The method according to claim 5, wherein said one or more signs of skin aging includes skin sagging or atrophy.

8. The method according to claim 1, wherein said one or more signs of skin aging includes discoloration of the skin,

9. The method according to claim 1, wherein said one or more signs of skin aging includes loss of skin elasticity.

10. A method of preventing, treating or ameliorating wrinkles or fine lines in skin, comprising:
topically applying to said wrinkle or fine lines a composition comprising an extract o Derris scandals Benth. and a cosmetically acceptable vehicle.

11. A topical composition for providing an anti-aging benefit to skin comprising:
an amount of an extract of Derris scandens Benth. effective to provide an anti-aging benefit to the skin; and
a cosmetically acceptable vehicle;
said topical composition being in the form of a lotion, cream, ointment, gel, or stick.

12. The composition of claim 11, wherein said extract is present in an amount sufficient to increase expression of LOXL1, inhibit calcineurin activity, or stimulate glycosaminoglycan synthesis.

13. The composition of claim 11, wherein said extract is present in an amount from 0.0001 weight % to 90 weight % based on total weight of the composition.

14. The composition of claim 13, wherein said extract is present in an amount from 0.001 weight % to 25 weigh based on the total weight of the composition.

15. The composition of claim 14, wherein said extract is present in an amount from 0,01 weight % to 10 weight % based on the total weight of the composition.

16. The composition of claim 11, wherein the extract is in combination with at least one other skin active.

17. The composition of claim 11, wherein the extract is in combination with at least one other botanical.

18. The composition of claim 17, wherein the other botancial comprises an extract from Butea Frondosa.
